(19) United States
(12) Patent Application Publication
(10) Pub. No.: US 2026/0083344 A1
(43) Pub. Date: Mar. 26, 2026

(54) GUT BARRIER ASSESSMENT UTILIZING CONTRAST AGENT

(71) Applicant: Motilent Limited, London (GB)

(72) Inventor: Alexander Menys, London (GB)

(21) Appl. No.: 19/329,840

(22) Filed: Sep. 16, 2025

Related U.S. Application Data
(60) Provisional application No. 63/696,976, filed on Sep. 20, 2024.

(51) Int. Cl. A61B 5/055; A61B 5/00; G01R 33/56

(52) U.S. Cl. CPC A61B 5/055; A61B 5/4255; G01R 33/5601

(57) ABSTRACT

Methods and systems for assessing gut barrier function with medical imaging techniques and orally ingested contrast are discussed herein. For example, a method for assessing gut health of a patient includes administering a contrast agent to the patient. The method further includes performing an magnetic resonance imaging (MRI) scan of the patient. The method further includes determining a metric corresponding to the contrast agent in a gut of the patient based on the performed MRI scan. The method further includes assessing health of the gut of the patient based on the metric corresponding to the contrast agent in the gut of the patient.

### CLAIM OF PRIORITY

The present application claims priority to United State Provisional Patent Application Number 63/696,976 filed on Sep. 20, 2024 and titled “Gut Barrier Assessment Utilizing Contrast Agent,” which is herein incorporated by reference in its entirety.

### FIELD

The present disclosure relates generally to systems and methods for assessing gut health.

### BACKGROUND

The intestinal barrier is a semi permeable structure that provides a protective role for the body keeping out harmful microorganisms and antigens. Specifically, the epithelial barrier and vascular barrier along with proper function of tight junctions is an important aspect of good gut health. In a number of diseases including, for example, celiac disease and inflammatory bowel disease (IBD) as well as human immunodeficiency virus (HIV) and/or acquired immunodeficiency syndrome (AIDS), following chemotherapy and radiotherapy and chronic use of non-steroidal anti-inflammatory drugs (NSAIDS) the gut barrier function can become degraded and even break down. The subsequent translocation of, for example, bacteria into the gut wall is thought to drive diseases like IBD especially in susceptible regions around the ileocecal valve and terminal ileum. Translocation to the mesentery, hepatic blood vessels and liver is thought to contribute to cirrhosis. Collectively, poor gut barrier function may cause excessive morbidity in the population. In current medical systems, there are several tests to assess gut barrier function including the mannitol ingestion test however such tests are highly variable and difficult to quantify.

### SUMMARY

A method for assessing gut health of a patient is disclosed. In one embodiment, the method includes administering a contrast agent to the patient; performing an MRI scan of the patient; determining a metric corresponding to the contrast agent in a gut of the patient based on the performed MRI scan; and assessing health of the gut of the patient based on the metric corresponding to the contrast agent in the gut of the patient.

Optionally, in some embodiments, assessing the health of the gut of the patient further comprises determining a gut barrier function of the gut of the patient.

Optionally, in some embodiments, the metric corresponding to the contrast agent comprises an absorption of the contrast agent in the gut of the patient.

Optionally, in some embodiments, the method further includes determining a period of time between administering the contrast agent to the patient and performing the MRI scan, wherein determining the metric is further based on the period of time.

Optionally, in some embodiments, the method further includes determining that the metric corresponding to the contrast agent in the gut is invalid, and in response, performing a second MRI scan of the gut of the patient.

Optionally, in some embodiments, determining the metric further comprises extracting a time of peak contrast and differential uptake from the MRI scan.

Optionally, in some embodiments, the method further includes administering a plurality of additional contrast agents to the patient prior to performing the MRI scan.

Optionally, in some embodiments, the metric corresponding to the contrast agent further comprises a contrast concentration of the contrast agent in the gut of the patient.

Optionally, in some embodiments, the metric corresponding to the contrast agent further comprises one or more of a presence of the contrast agent in solid organs and extramural spaces, a peak enhancement, a time to peak and a liver-kidney ratio.

Optionally, in some embodiments, assessing the health of the gut of the patient further comprises one or more of assessing an appearance of damage, assessing a presence of damage and dynamics of damage.

### BRIEF DESCRIPTION OF THE SEVERAL VIEWS OF THE DRAWINGS

FIG. 1 illustrates a simplified schematic of a system for assessing gut health, according to embodiments herein.

FIG. 2A illustrates an example anatomical image such as a magnetic resonance imaging (MRI) scan result of a patient prior to ingesting a contrast agent, according to embodiments herein.

FIG. 2B illustrates an example anatomical image such as an MRI scan result of a patient after ingesting a contrast agent, according to embodiments herein.

FIG. 3 is a simplified block diagram of components of a computing system 300 of the system of FIG. 1 according to embodiments herein.

### DETAILED DESCRIPTION

Embodiments herein introduce details on the use of an oral contrast agent visible with magnetic resonance imaging (MRI). For example, a molecule of a known size (or sizes) is ingested by a patient as part of a liquid or solid meal. Uptake of the contrast is possible where there is degradation of the gut barrier and the effects of the contrast agent can be seen using appropriate imaging with MRI.

In some embodiments, the patient may ingest a gadolinium contrast agent. The paramagnetic effects of gadolinium change the susceptibility of water protons making them visible on T1 weighted MRI imaging. As gadolinium cannot penetrate the healthy gut barrier (e.g., at levels of around 600 grams/mole) it should remain contained in the lumen in healthy individuals. Where the barrier is permeable the gadolinium contrast agent can move freely into, for example, the duodenum and proximal small bowel where it can subtly enhance the liver.

In some embodiments, susceptibility mapping and quantification techniques may be used to quantify and track the gadolinium contrast agent absorption thus being able to assess gut health. For example, the concentration of contrast can be calculated as well as update ratios within the liver and between liver, kidneys and bladder enabling pharmacodynamic assessment. Alternatively or in addition, different sizes of contrast-conjugated orally ingested mediums may be ingested over time to establish the size of the barrier gaps. In some cases, a secondary agent may be ingested that binds to tight junctions or other ligands to provide a coverage of degradation over the bowel surface.

In some instances, the agent may be administered as an enema to interrogate the distal bowel function including, particularly important in, for example, Ulcerative Colitis and bacterial infections of the like.

In some embodiments a protocol may be introduced for assessing gut health (gut barrier function) using a contrast agent.

The protocol may begin with the patient fasting (or being fasted) before the start of the MRI exam (“Step 1”). An oral contrast is prepared with the agent mixed thoroughly with a suitable liquid depending on the nature of the suspected GI disruption (“Step 2”). The patient ingests the contrast containing liquid over a certain amount of time (e.g., 15 minutes) before entering the MRI scanning room. The time of ingestion is recorded (“Step 3”). The patient lies on the MRI scanner bed and the body coil is placed over the patient (“Step 4”). The MRI technician performs planning scans and a set of sequences specifically designed to image contrast enhanced tissue (e.g., a T1W MRI scanning protocol) (“Step 5”). The patient is scanned once or more than once to establish a suitable uptake curve (“Step 6”). Imaging data is then reviewed for quality and read by a suitably qualified medical professional (“Step 7”).

Extraction of T1 signal is performed by placing one or more regions of interest on, for example, the liver, kidney, and other organs where contrast was taken up (“Step 8”). The time of peak contrast and differential uptake is extracted from the image time stamp (“Step 9”). Key metrics for barrier function include, for example, the presence of contrast in solid organs and extramural space, peak enhancement, time to peak and/or a liver-kidney ratio. Key metrics for mucosal damage include, for example, appearance of damage (e.g., patchy, diffuse, proximal, distal), the presence of damage, and/or dynamics of damage (e.g., penetrating, mucosal or transmucosal). The metrics are tabulated and, optionally, curves of the metrics may be created if/when needed by, for example, a medical expert using a computing system (or by artificial intelligence/machine learning models stored/deployed at the computing system or at a server). In some cases, the medical expert may review the metrics (or curves) to assess the gut health of the patient.

In some embodiments, a follow up scan may be performed to assess the efficacy of various treatments recommended or taken. In some cases, the treatments may be recommended based on the metrics of the performed MRI. For example, a follow up scan may be performed to assess the cessation of NSAIDs or removal of gluten from the diet.

It should be understood that the protocol according to embodiments herein may be repeated with various different contrast agent(s) with different properties to interrogate other aspects of mucosal damage or other damage that the gut of the patient may have.

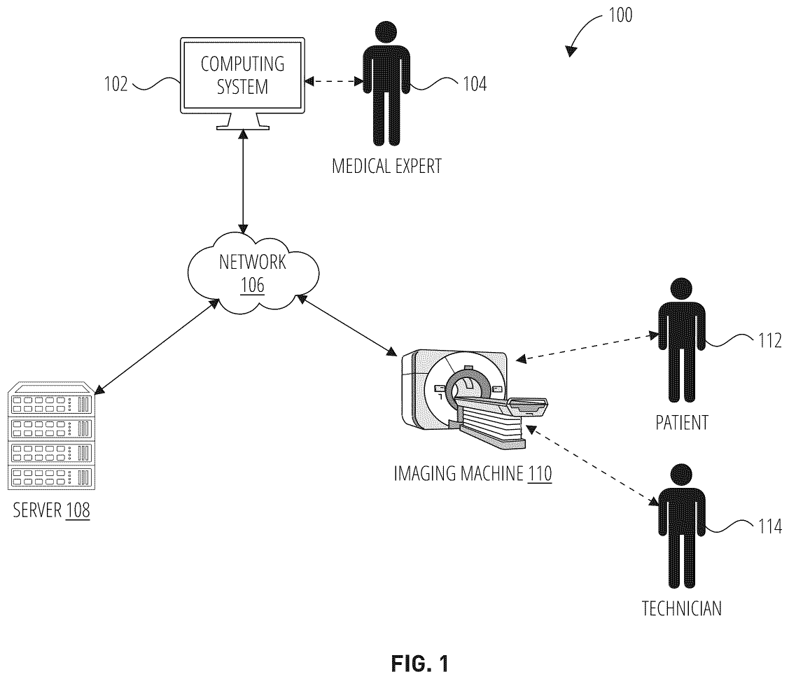

Turning to the figures, FIG. 1 illustrates a simplified schematic of a system 100 for assessing gut health, according to embodiments herein.

The system 100 used to perform or assist in performing any of the embodiments discussed herein includes a server 108, a network 106, an imaging machine 110, such as an MRI machine which a patient 112 and/or a technician 114 may interact with, and a computing system 102 which a medical expert 104 may interact with.

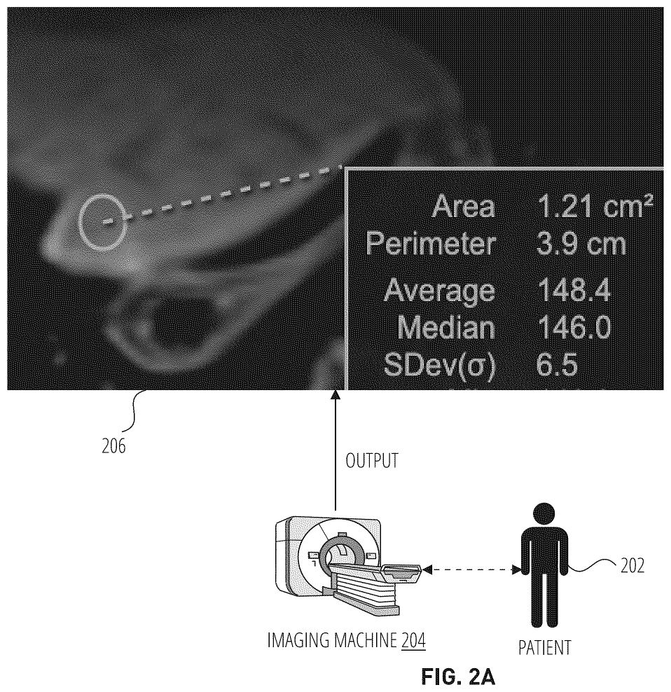

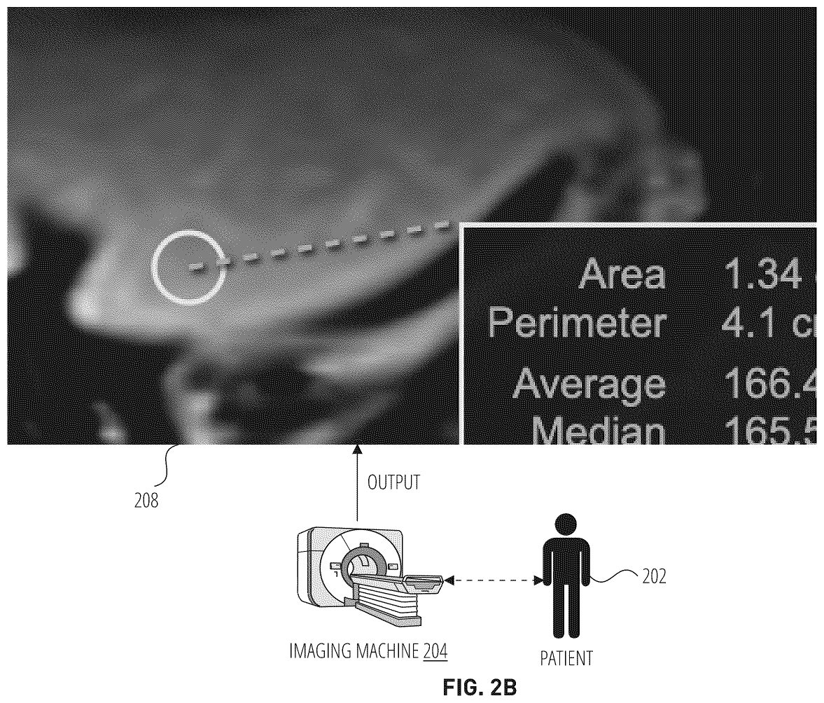

FIG. 2A illustrates an example anatomical image 206 such as an MRI scan result of a patient 202 prior to ingesting a contrast agent, according to embodiments herein.

In some embodiments, optionally, the patient 202 may have an MRI (e.g., imaging machine 204) scan performed on their gut (i.e., their stomach) prior to ingesting a contrast agent as to obtain a baseline of the patient's stomach. For example, the patient 202, prior to ingesting any agent, may have an average contrast signal strength of 148.4 for their stomach.

FIG. 2B illustrates an example anatomical image 208 such as an MRI scan result of a patient 202 after ingesting a contrast agent, according to embodiments herein.

In some embodiments, once a patient 202 has ingested a contrast agent, an MRI (e.g., imaging machine 204) scan may be performed with a focus on the patient's gut (i.e., stomach). As discussed herein, in some embodiments, the MRI technician performs planning scans and a set of sequences specifically designed to image contrast enhanced tissue (e.g., T1W) (“Step 5”). The patient 202 is scanned once or more than once to establish a suitable uptake curve (“Step 6”). Imaging data is then reviewed for quality and read by a suitably qualified medical professional (“Step 7”). Extraction of T1 signal is performed by placing one or more regions of interest on, for example, the liver, kidney, and other organs where contrast was taken up (“Step 8”). The time of peak contrast and differential uptake is extracted from the image time stamp (“Step 9”). Key metrics for barrier function include, for example, the presence of contrast in solid organs and extramural space, peak enhancement, time to peak and/or a liver-kidney ratio. Key metrics for mucosal damage include, for example, appearance of damage (e.g., patchy, diffuse, proximal, distal), the presence of damage, and/or dynamics of damage (e.g., penetrating, mucosal or transmucosal). The metrics are tabulated and optionally curves may be created if/when needed.

In some embodiments, the MRI scan result is quantified and the strength of the contrast signal after administering the contrast agent has a higher signal strength (e.g., 166.4). Thus, a portion of the contrast agent has absorbed by the gut barrier indicating that the gut barrier is not fully healthy. In some instances, the patient may be advised of options to remedy and/or mitigate the gut barrier problems. Embodiments herein may assess the gut health of the patient with higher accuracy when the patient ingests the contrast agent before undergoing MRI scans.

### EXAMPLE EMBODIMENTS

Certain embodiments disclosed herein systematically record data before, during and after ingestion of a contrast agent.

Certain embodiments disclosed herein display relevant images along with priors.

Certain embodiments disclosed herein place systematic measurements of signal intensity within the MRI dataset.

Certain embodiments disclosed herein standardize region of interest (ROI) placement, size, and location in 3D space.

Certain embodiments disclosed herein interpolate and collate measurements to produce absolute values or curves for comparison.

Certain embodiments disclosed herein markup location(s) of gut damage in the patient.

Certain embodiments disclosed herein collate and report values of gut permeability.

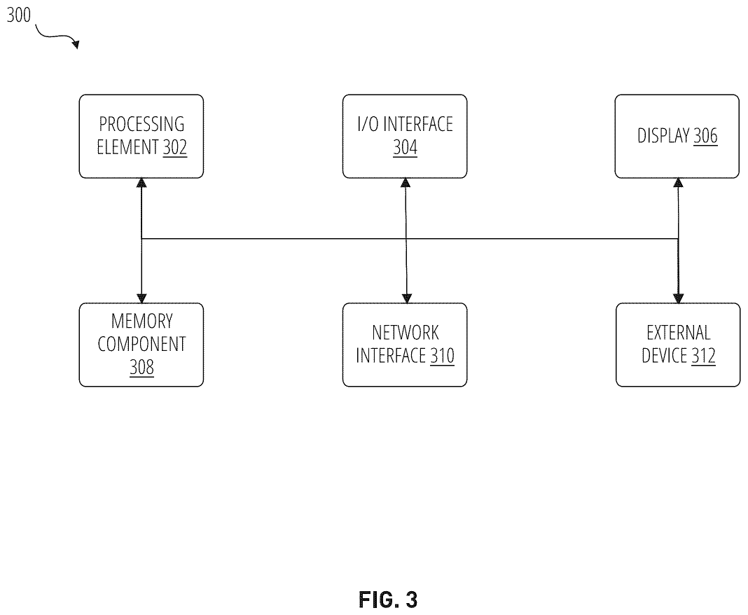

FIG. 3 is a simplified block diagram of components of a computing system 300 of the system of FIG. 1 according to embodiments herein. For example, the processing element 302 and the memory component 308 may be located at one or in several computing systems 300. This disclosure contemplates any suitable number of such computing systems 300. For example, the server may be a desktop computing system, a mainframe, a blade, a mesh of computing systems 300, a laptop or notebook computing system 300, a tablet computing system 300, an embedded computing system 300, a system-on-chip, a single-board computing system 300, or a combination of two or more of these. Where appropriate, a computing system 300 may include one or more computing systems 300; be unitary or distributed; span multiple locations; span multiple machines; span multiple data centers; or reside in a cloud, which may include one or more cloud components in one or more networks. A computing system 300 may include one or more processing elements 302, an input/output I/O interface 304, one or more external devices 312, one or more memory components 308, and a network interface 310. Each of the various components may be in communication with one another through one or more buses or communication networks, such as wired or wireless networks. The components in FIG. 3 are exemplary only. In various examples, the computing system 300 may include additional components and/or functionality not shown in FIG. 3.

The processing element 302 may be any type of electronic device capable of processing, receiving, and/or transmitting instructions. For example, the processing element 302 may be a central processing unit, microprocessor, processor, or microcontroller. Additionally, it should be noted that some components of the computing system 300 may be controlled by a first processing element 302 and other components may be controlled by a second processing element 302, where the first and second processing elements may or may not be in communication with each other.

The I/O interface 304 allows a user to enter data in to computing system 300, as well as provides an input/output for the computing system 300 to communicate with other devices or services. The I/O interface 304 can include one or more input buttons, touch pads, touch screens, and so on.

The external device 312 are one or more devices that can be used to provide various inputs to the computing systems 300, e.g., mouse, microphone, keyboard, trackpad, sensing element (e.g., a thermistor, humidity sensor, light detector, etc. The external devices 312 may be local or remote and may vary as desired. In some examples, the external devices 312 may also include one or more additional sensors.

The memory components 308 are used by the computing system 300 to store instructions for the processing element 302, as well as store data. The memory components 308 may be, for example, magneto-optical storage, read-only memory, random access memory, erasable programmable memory, flash memory, or a combination of one or more types of memory components.

The network interface 310 provides communication to and from the computing system 300 to other devices. The network interface 310 includes one or more communication protocols, such as, but not limited to Wi-Fi, Ethernet, Bluetooth, etc. The network interface 310 may also include one or more hardwired components, such as a Universal Serial Bus (USB) cable, or the like. The configuration of the network interface 310 depends on the types of communication desired and may be modified to communicate via Wi-Fi, Bluetooth, etc.

The display 306 provides a visual output for the computing system 300 and may be varied as needed based on the device. The display 306 may be configured to provide visual feedback to a user and may include a liquid crystal display screen, light emitting diode screen, plasma screen, or the like. In some examples, the display 306 may be configured to function as an input element for a user through touch feedback or the like.

Any description of a particular component being part of a particular embodiment, is meant as illustrative only and should not be interpreted as being required to be used with a particular embodiment or requiring other elements as shown in the depicted embodiment.

All relative and directional references (including top, bottom, side, front, rear, and so forth) are given by way of example to aid the reader's understanding of the examples described herein. They should not be read to be requirements or limitations, particularly as to the position, orientation, or use unless specifically set forth in the claims. Connection references (e.g., attached, coupled, connected, joined, and the like) are to be construed broadly and may include intermediate members between a connection of elements and relative movement between elements. As such, connection references do not necessarily infer that two elements are directly connected and in fixed relation to each other, unless specifically set forth in the claims.

The present disclosure teaches by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall there between.

1. A method for assessing gut health of a patient comprising:
administering a contrast agent to the patient;
performing an MRI scan of the patient;
determining a metric corresponding to the contrast agent in a gut of the patient based on the performed MRI scan; and
assessing health of the gut of the patient based on the metric corresponding to the contrast agent in the gut of the patient.

2. The method of claim 1, wherein assessing the health of the gut of the patient further comprises determining a gut barrier function of the gut of the patient.

3. The method of claim 1, wherein the metric corresponding to the contrast agent comprises an absorption of the contrast agent in the gut of the patient.

4. The method of claim 1, further comprising:
determining a period of time between administering the contrast agent to the patient and performing the MRI scan; and
wherein determining the metric is further based on the period of time.

5. The method of claim 1, further comprising:
determining that the metric corresponding to the contrast agent in the gut is invalid; and
in response, performing a second MRI scan of the gut of the patient.

6. The method of claim 1, wherein determining the metric further comprises extracting a time of peak contrast and differential uptake from the MRI scan.

7. The method of claim 1, further comprising:
administering a plurality of additional contrast agents to the patient prior to performing the MRI scan.

8. The method of claim 1, wherein the metric corresponding to the contrast agent further comprises a contrast concentration of the contrast agent in the gut of the patient.

9. The method of claim 1, wherein the metric corresponding to the contrast agent further comprises one or more of a presence of the contrast agent in solid organs and extramural spaces, a peak enhancement, a time to peak and a liver-kidney ratio.

10. The method of claim 1, wherein assessing the health of the gut of the patient further comprises one or more of assessing an appearance of damage, assessing a presence of damage and dynamics of damage.

11. A method for analyzing gut health comprising:
receiving first images corresponding to a gut of the patient a first time duration after ingestion of a contrast agent;
determining a metric corresponding to the contrast agent in the gut of the patient based on the first images; and
assessing health of the gut of the patient based on the metric corresponding to the contrast agent in the gut of the patient.

12. The method of claim 11, wherein assessing the health of the gut of the patient further comprises determining a gut barrier function of the gut of the patient.

13. The method of claim 11, wherein the metric corresponding to the contrast agent comprises an absorption of the contrast agent in the gut of the patient.

14. The method of claim 11, wherein determining the metric is further based on the first time duration.

15. The method of claim 11, further comprising:
determining that the metric corresponding to the contrast agent in the gut is invalid; and
in response, requesting second images corresponding to the gut of the patient.

16. The method of claim 11, wherein determining the metric further comprises extracting a time of peak contrast and differential uptake from the first images.

17. The method of claim 11, wherein the metric corresponding to the contrast agent further comprises a contrast concentration of the contrast agent in the gut of the patient.

18. The method of claim 11, wherein the metric corresponding to the contrast agent further comprises one or more of a presence of the contrast agent in solid organs and extramural spaces, a peak enhancement, a time to peak and a liver-kidney ratio.
